(19) United States
(12) Patent Application Publication
(10) Pub. No.: US 2005/0226886 A1
(43) Pub. Date: Oct. 13, 2005

(54) Preferential detection of procarboxypeptidase R (thrombin activatable fibrinolyisis inhibitor) by enzyme-linked immunosorbent assay

(76) Inventor: Hidechika Okada, Nagoya (JP)

Correspondence Address: GRAYBEAL JACKSON HALEY LLP, Suite 350, 155-108th Avenue N.E., Bellevue, WA, 98004-5973, US

(21) Appl. No.: 11/084,461

(22) Filed: Mar. 17, 2005

Related U.S. Application Data
(60) Provisional application No. 60/554,741, filed on Mar. 18, 2004.

(51) Int. Cl. A61K 39/00

(52) U.S. Cl. 424/184.1

(57) ABSTRACT

Antibodies that specifically recognize mammalian carboxypeptidase molecules are provided which are useful in diagnostic and therapeutic methods. Exemplary monoclonal antibodies (mAbs) specifically bind to pro-carboxypeptidase R (proCPR), also known as thrombin activatable fibrinolysis inhibitor (TAFI). These mAbs are useful in immunoassays, including an exemplary, sandwich enzyme-linked immunosorbent assay (ELISA) system, to detect proCPR. Since the amount of the antigen detectable by the ELISA was essentially the same in fresh plasma and serum incubated at 37° C. for 1 hr, we concluded that the ELISA system detected not only proCPR but also inactivated CPR generated from proCPR. However, an appreciable amount of proCPR remained unactivated in serum. For extensive activation of proCPR in plasma, thrombin and thrombomodulin complexes (T-TM) together with CaCl₂ can be used. Following extensive conversion of proCPR to CPR by T-TM and CaCl₂, converting plasma to serum (T-TM serum), antigenicity became undetectable by ELISA. Further analysis revealed that 2A16 reacts only with proCPR although 10G1 reacts with proCPR, active CPR and inactivated CPR. Therefore, we concluded that the ELISA system preferentially detects proCPR and not CPR. Our sandwich ELISA system utilizing 2A16 and 10G1 provides a suitable method for detecting proCPR and can be used to determine levels of proCPR in plasma samples from patients.

### CROSS-REFERENCE TO RELATED APPLICATIONS

The present application claims priority from U.S. Provisional Patent application 60/554,741, filed Mar. 18, 2004.

### TECHNICAL FIELD

The instant invention relates to immunological methods and compositions. More specifically, the invention relates to antibodies that bind carboxypeptidases.

### BACKGROUND OF THE INVENTION

Anapylatoxins such as C3a, C4a, and C5a, which are generated during complement activation, have arginine at their carboxyl terminal (C-terminal). The physicochemical and physiological properties of C3a, C4a and C5a, termed anaphylatoxins, are known. Each is a potent bioactive polypeptide and plays a key role as a mediator of acute inflammatory processes. Among the three anaphylatoxins, C5a is characterized by its ability to interact with white blood cells. C3a and C4a are rendered spasmogenically inactive in vivo by conversion of the respective des arginine derivatives (C3a des Arg or C3ai, C4ai des Arg or C4ai) by a serum carboxypeptidase. Human C5a is converted to C5a des Arg by a serum carboxypeptidase.

Anaphylatoxin products have been implicated in various naturally occurring pathologic states including: autoimmune disorders such as systemic lupus erythematosus, rheumatoid arthritis, malignancy, myocardial infarction, Purtscher's retinopathy, sepsis and adult respiratory distress syndrome. In addition, increased circulating levels of C3a and C5a have been detected in conditions associated with iatrogenic complement activation such as: cardiopulmonary bypass surgery, renal dialysis, and nylon fiber leukaphoresis. Elevated levels of C4a anaphylatoxin is associated with the autoimmune disorders mentioned above.

Activated fragments of complement proteins include C3a, C4a, C5a anaphylatoxins, and C5b-9 membrane attack complexes. These fragments mediate several functions including leukocyte chemotaxis, activation of macrophages, vascular permeability and cellular lysis (Frank, M. and Fries, L. Complement. In Paul, W. (ed.) Fundamental Immunology, Raven Press, 1989).

Carboxypeptidases are important mediators of activity of human complement systems, particularly with regard to regulation of anaphylatoxin products. Removal of the C-terminal arginine by a basic carboxypeptidase (CPB), such as carboxypeptidase N (CPN) diminishes anaphylatoxin activity (See Bokisch, et al. (1970) J. Clin. Invest. 49: 2427-2436.) Another CPB termed carboxypeptidase R (CPR) was found in fresh serum (See Campbell, et al. (1989) Biochem. Biophys. Res. Commun. 162: 933-939) in addition to CPN, (See Erdos, et al. (1965) Clin. Chim. Acta II: 39-43); Plummer Jr., et al.(1978) J. Biol. Chem. 253: 3907-3912), which had been thought to be the only CPB present in plasma and serum. CPR was also reported independently by others who termed it carboxypeptidase U (See Hendriks, et al. (1989) J. Clin. Chem. Clin. Biochem. 27: 277-285; Hendriks, et al. (1990) Biochim Biophys Acta 1034: 86-92), and plasma CPB (See Eaton, et al. (1991) J. Biol. Chem. 266: 21833-21838.)

CPR is generated from its zymogen (proCPR) by proteolytic enzymes such as trypsin, thrombin and plasmin (See Campbell, et al. (1970) J. Lab. Clin. Med. 115: 610-642; Eaton, et al. (1991) J. Biol. Chem. 266: 21833-21838; Shinohara, et al. (1994) Int. Arch. Allergy. Immunol. 103: 400-404.) ProCPR is also converted to CPR by neutrophil elastase (See Kawamura, et al. (2002) Microbiol. Immunol. 46: 225-230.) Although CPR was shown to be a possible inactivator of bioactive peptides such as C3a, C5a and bradykinine (See Campbell, et al. (2002) Microbiol. Immunol. 46: 131-134; Campbell, et al. (2001) Immunol. Rev. 180: 162-167; Shinohara, et al. (1994) Int. Arch. Allergy. Immunol. 103: 400-404.) ProCPR turned out to be the same molecule as thrombin activatable fibrinolysis inhibitor (TAFI) (See Bajzar, et al. (1995) J. Biol. Chem. 270: 14477-14484) with CPR corresponding to activated TAFI (TAFIa) which removes C-terminal lysine from fibrin and degraded fibrin resulting in interference in the binding of plasminogen to lysine residues on fibrin for its activation by tissue plasminogen activator (t-PA) (See Bajzar, et al. (1996) J. Biol. Chem. 271: 16603-16608; Redlitz, et al. (1995) J. Clin. Invest. 96: 2534-2538.) Therefore, proCPR plays an important role not only in restriction of inflammation but also in regulation of fibrinolysis.

Therefore, the ability to modulate circulating levels of these anaphylatoxins or their des-Arg derivatives would be of utility in managing and treating a variety of important pathological conditions. Additionally, the ability to measure levels of C4a and C4a des Arg permits determination of the pathway by which complement activation occurs, thereby permitting a determination of the precise mechanism of complement activation and also whether natural immunological defense mechanisms are functional.

Previously, we developed monoclonal antibodies (mAbs) against proCPR and established an enzyme-linked immunosorbent assay (ELISA) system using the two mAbs to determine the amount of proCPR and/or CPR (See Guo, et al. (1999) Microbiol. Immunol. 43: 691-698.) Since the amounts detected in plasma and serum heated at 37° C. were essentially the same, we assumed that the ELISA system would detect proCPR, activated CPR and inactivated CPR, and we regarded the amount detected by the ELISA system as CPR-total (See Guo, et al. (1999) Microbiol. Immunol. 43: 691-698.)

However, it was recently discovered that only a portion of proCPR is activated during conversion of plasma to serum and that an appreciable amount of proCPR is present in serum. For extensive activation of proCPR in plasma, thrombin and thrombomodulin complexes (T-TM) can be used (See Bajzar, et al. (1996) J. Biol. Chem. 271: 16603-16608; Hosaka, et al. (1998) Thromb. Haemost. 79: 371-377.)

Therefore, we evaluated with ELISA the amount of antigens detected in plasma before and after activation by T-TM.

### SUMMARY OF EXEMPLARY EMBODIMENTS OF THE INVENTION

The present invention provides novel antibodies that specifically bind carboxypeptidase molecules, as well as related compositions and methods employing these antibodies. In more detailed embodiments, the antibodies of the invention specifically bind to one or more carboxipepsidase R (CPR) molecules selected from proCPR, activated CPR, and inactivated CPR. In certain detailed embodiments, an antibody of the invention binds to a specific form of CPR, such as pro-CPR.

The invention further provides immunoassay methods for detecting a presence or quantity of proCPR, activated CPR, and/or inactivated CPR in a test sample or subject. These assay methods include contacting an antibody reagent that detects proCPR, activated CPR, and/or inactivated CPR with a test sample, incubating the antibody and test sample to allow antibody binding to proCPR, activated CPR, and/or inactivated CPR in the sample, and detecting the antibody binding to the proCPR, activated CPR, and/or inactivated CPR to indicate the presence or quantity of proCPR, activated CPR, and/or inactivated CPR in the sample.

In related embodiments, the invention provides methods for diagnosis and treatment of mammalian subjects having, or at risk of having, a CPR-associated fibrinolytic or inflammatory disorder. The CPR-associated fibrinolytic disorder or CPR-associated inflammatory disorder is marked by aberrant expression, metabolism, or activity of an endogenous CPR (proCPR, activated CPR, and/or inactivated CPR) in the subject. Among the disorders targeted for diagnosis and treatment by the invention are various symptoms and conditions associated with viral and other parasitic infections, tissue injury, organ transplant rejection, autoimmune diseases, and a diverse array of inflammatory responses (e.g., inflammatory responses associated with Alzheimer's disease or bacterial infection). The methods for detecting such disorders generally include contacting an anti-CPR antibody of the invention with a sample from the subject. The antibody binds to the proCPR, activated CPR, and/or inactivated CPR in the subject, and this binding is detected, qualitatively or quantitatively, to diagnose the CPR-associated fibrinolytic or inflammatory disorder.

Also provided herein are kits comprising an anti-proCPR, activated CPR, and/or inactivated CPR antibody in combination with one or more additional reagent(s), or device(s) useful for detecting the presence or quantity of proCPR, activated CPR, and/or inactivated CPR in a sample.

### BRIEF DESCRIPTION OF THE DRAWINGS

FIG. 1 provides sandwich ELISA values for plasma and serum employing the concepts of the invention. From blood samples of each of 3 individuals, we prepared citrated plasma and serum by incubating at 37° C. for 2 hr and at 4° C. overnight. Fifty microliters of plasma and serum diluted 1:200 in 50 mM Tris-HCl buffered saline (TBS) were incubated in each well of a 2A 16-coated plate at room temperature for 1 hr. After washing the plate 3 times with 300 μl/well of PBS-T, each well was treated with 50 μl HRP-10G1 (0.2 μg/ml) and incubated at room temperature for 1 hr before washing 3 times with PBS-T. Then, 100 μl of OPD-H₂O₂ were added to each well to detect bound HRP-10G1 10 min before the addition of 3N H₂SO₄ to stop the reaction. The OD₄₉₀ of each well was determined on a plate reader. The values of sera were higher than those of plasma.

FIG. 2 provides results of ELISA employing the concepts of the invention with varying sample dilutions. Plasma, 2 hr at 25° C. T-TM serum and 2 hr at 37° C. serum were incubated on the ELISA plate at 1/25, 1/50, 1/100, and 1/200 dilutions. T-TM sera incubated at 25° C. or 37° C. showed no immunoreactivity on ELISA even at a 1/25 dilution. Since active CPR remained to some extent (about 30% as shown in FIG. 6B) in T-TM serum incubated at 25° C. for 2 hr, the ELISA system did not detect active or inactivated CPR.

FIG. 3 illustrates the effect of complete conversion of proCPR to CPR by T-TM. Plasma samples were prepared by mixing 1 μl plasma with 179 μl of TBS and 20 μl of PPACK (25 μg/ml) resulting in 1/200 diluted plasma. Ten minute T-TM sera were prepared by incubating 1 μl of plasma with 4 μl of thrombin (2 units/ml) containing CaCl₂ (100 mM) and 16 μl of thrombomodulin (20 ng/ml) at 25° C. for 10 min before addition of 20 μl of 25 μg/ml PPACK to inhibit thrombin. Two hour T-TM sera were prepared by incubating 1 μl of plasma with 4 μl of thrombin (2 units/ml) containing CaCl₂ (100 mM) and 16 μl of thrombomodulin (20 μg/ml) at 37° C. for 2 hr before addition of 20 μl of 25 μg/ml PPPACK to inhibit thrombin. Each sample was subjected to the sandwich ELISA. Although the values for the 10-min T-TM serum varied among the samples, those of the 2-hr T-TM sera were generally extremely low.

FIG. 4 illustrates the effect of preincubation of 10G1 with T-TM serum. Plasma, 5-min T-TM serum and 1-hr T-TM serum (final dilution, 1/200) were prepared in a same manner as described in the legend for FIG. 3. HRP-10G1 (0.2 μg/ml) was mixed with a 1/200 dilution of plasma, 5-min T-TM serum or 1-hr T-TM serum and incubated for 7 hr at room temperature. The preincubated HRP-10G1 were subjected to determination of reactivity with proCPR captured on an ELISA plate which was coated with 2 μg/ml 2A16 to limit the capacity and incubated with plasma for 7 hr at room temperature before washing to remove unbound proteins. The binding of 10G1 to proCPR on the ELISA plate was inhibited with 1-hr T-TM serum as well as with plasma and 5-min T-TM serum.

FIG. 5 illustrates the effect of preincubation of the 2A16-coated plate with T-TM serum. To wells of an ELISA plate coated with 2 μg/ml 2A16, various dilutions (1/25, 1/50, 1/100, 1/200 and TBS alone) of plasma, 25° C. for 2 hr T-TM serum or 37° C. for 2 hr T-TM serum were added. After incubation at 4° C. overnight, the plate was washed to remove unbound proteins. To each well, 50 μl of 1/5000 HRP-10G1 preincubated with an equal volume of 1/200 plasma were added. Even at a 1/25 dilution, pretreatment of the plate with 37° C. for 2 hr T-TM serum did not inhibit the binding of HRP-10G1 mixed with plasma, indicating that CPR in the T-TM serum did not interfere with the binding of proCPR bound to HRP-10G1. The incomplete inhibition by plasma may have been due to a relative excess of 2A16 on the plate or the presence of free HRP-10G1 to some extent.

FIG. 6 demonstrates changes in immunoreactivity with the ELISA and in the CPR activity of T-TM serum following incubation at 25° C. (A) Loss of immunoreactivity during activation of proCPR by T-TM. Plasma at dilutions of 1/25 and 1/50 was mixed with T-TM and CaCl₂, and incubated at 25° C. After the indicated incubation period, PPACK (final concentration: 2.5 μg/ml) was added to stop thrombin activity and subjected to determination of immunoreactivity in our ELISA system. The immunoreactivity of proCPR in the plasma promptly decreased following addition of T-TM and CaCl₂. (B) CPR activity in T-TM serum following incubation at 25° C. CPR activity was determined by cleavage of Hip-Arg as a synthetic substrate (See Hendriks, et al. (1985) Clin. Chem. 31: 1936-1939.) CPR activity remained at a high level at 30 min and approximately 30% of the maximum activity remained even after 120 min of incubation at 25° C. “Pre” indicates the plasma before addition of T-TM.

### DETAILED DESCRIPTION OF EXEMPLARY EMBODIMENTS OF THE INVENTION

As noted above, the instant invention provides new and useful antibodies directed against carboxypeptidase molecules. The antibodies of the invention specifically bind to one or more carboxipepsidase R (CPR) molecules selected from proCPR, activated CPR, and inactivated CPR.

As used herein, “antibody” represents an immunoglobulin protein which is capable of binding an antigen. An antibody can include the entire antibody, as well as any antibody fragments (e.g., F(ab′, Fab, Fv) capable of binding the epitope, antigen or antigenic fragment of interest (see below). Preferred antibodies for assays of the invention are immnunoreactive or immunospecific for, and therefore specifically and selectively bind to, proCPR, activated CPR, and/or inactivated CPR.

The antibodies of the invention bind to one or more carboxipepsidase R (CPR) molecules selected from proCPR, activated CPR, and inactivated CPR. This CPR binding activity is specific, which means that the observed binding of antibody to CPR is not substantially blocked by non-specific reagents (e.g., by non-specific interactions with unrelated proteins, such as BSA). In more detailed embodiments, antibody specificity is such that the antibody will bind CPR, but not other mammalian carboxypeptidase molecules (e.g., CPN). For example, an antibody of the invention may be contacted with, and bind with high or moderate affinity to, one or more forms of CPR—and the resulting binding interaction will not be significantly blocked or reduced by competitive binding when a different carboxypeptidase (e.g., CPN) is added. Alternatively, the antibody may not exhibit detectable binding against non-CPR molecules, including other carboxypeptidase molecules such as CPN.

In certain detailed embodiments, antibodies of the invention are capable of even more selective binding, such as by binding preferentially, or exclusively, to a specific form of CPR, such as pro-CPR, activated CPR, or inactivated CPR. This enhanced selectivity CPR binding activity is form specific, meaning the observed binding of antibody to a selected form of CPR is not substantially blocked by addition of another form of CPR. For example, a form-specific CPR antibody of the invention may be contacted with, and bind with high or moderate affinity to, a particular form of CPR, e.g., pro-CPR, and the resulting binding interaction will not be significantly blocked or reduced by competitive binding when a different “non-cognate” form of CPR (e.g., activated CPR, and/or inactivated CPR) is added. In certain exemplary embodiments, an antibody specifically binds pro-CPR, and does not exhibit detectable binding, or measurable competition, against one or more different forms of CPR, in some cases against multiple non-cognate forms of CPR.

As used herein, the term “antibody” encompasses all types of antibodies, e.g., polyclonal, monoclonal, and those produced by the phage display methodology. Particularly preferred antibodies of the invention are antibodies which have a relatively high degree of affinity for proCPR, activated CPR, and/or inactivated CPR. In certain embodiments, the antibodies will exhibit an affinity for proCPR, activated CPR, and/or inactivated CPR of about Kd<10^{-8 M}.

The phrase “substantially purified” when referring to anti-CPR antibodies of the present invention, means a composition which is essentially free of other cellular components with which the antibodies are associated in a non-purified, e.g., native state or environment. Purified antibody is generally in a homogeneous state, although it can be in either in a dry state or in an aqueous solution. Purity and homogeneity are typically determined using analytical chemistry techniques such as polyacrylamide gel electrophoresis or high performance liquid chromatography.

Generally, substantially purified anti-CPR antibody comprises more than 80% of all macromolecular species present in a preparation prior to admixture or formulation of the antibody with a pharmaceutical carrier, excipient, adjuvant, buffer, absorption enhancing agent, stabilizer, preservative, adjuvant or other co-ingredient. More typically, the antibody is purified to represent greater than 90% of all proteins present in a purified preparation. In specific embodiments, the antibody is purified to greater than 95% purity or may be essentially homogeneous wherein other macromolecular species are not detectable by conventional techniques.

Within more detailed embodiments, the invention also provides diagnostic and therapeutic antibodies, including monoclonal antibodies, and related compositions and methods for use in the diagnosis, management and treatment of disease. The antibodies specifically recognize one or more biomolecules selected from proCPR, activated CPR, and inactivated CPR, and are therefore useful for detecting and/or neutralizing these biomolecules, and/or blocking their interactions with other biomolecules, in vitro or in vivo.

The preparation of polyclonal antibodies is well-known to those skilled in the art. See, for example, Green et al, “Production of Polyclonal Antisera,” in: Immunochemical Protocols pages 1-5, Manson, ed., Humana Press 1992; Coligan et al, “Production of Polyclonal Antisera in Rabbits, Rats, Mice and Hamsters,” in: Current Protocols in Immunology, section 2.4.1, 1992. The preparation of monoclonal antibodies likewise is conventional. See, for example, Kohler & Milstein, 1975, Nature 256:495; and Harlow et al, in: Antibodies: a Laboratory Manual, page 726, Cold Spring Harbor Pub., 1988. The production of non-human monoclonal antibodies, e.g., murine or rat, can be accomplished by, for example, immunizing the animal with a preparation comprising purified proCPR, activated CPR, or inactivated CPR. The immunogen, often comprising a peptide/hapten complex or other conjugate as described herein, can be obtained from a natural source, by peptides synthesis, or by recombinant expression. Antibody-producing cells obtained from the immunized animals are immortalized and screened for the production of an antibody which binds to proCPR, activated CPR, and/or inactivated CPR (see, e.g., Harlow & Lane, Antibodies, A Laboratory Manual, CSHP, NY, 1988.

Humanized forms of mouse antibodies can be generated by linking the CDR regions of non-human antibodies to human constant regions by recombinant DNA techniques (see, e.g., Queen et al., Proc. Natl. Acad. Sci. USA 86:10029-10033, 1989 and WO 90/07861, each incorporated by reference). Human antibodies can be obtained using phage-display methods (see, e.g., Dower et al., WO 91/17271; McCafferty et al., WO 92/01047). In these methods, libraries of phage are produced in which members display different antibodies on their outersurfaces. Antibodies are usually displayed as Fv or Fab fragments. Phage displaying antibodies with a desired specificity may be selected by affinity enrichment. Human antibodies may be selected by competitive binding experiments, or otherwise, to have the same epitope specificity as a particular mouse antibody.

The invention further provides fragments of the intact antibodies described herein. Typically, these fragments compete with the intact antibody from which they were derived for specific binding to proCPR, activated CPR, and/or inactivated CPR. Antibody fragments include separate heavy chains, light chains Fab, Fab′ F(ab′)2, Fv, and single chain antibodies. Fragments can be produced by enzymic or chemical separation of intact immunoglobulins. For example, a F(ab′)2 fragment can be obtained from an IgG molecule by proteolytic digestion with pepsin at pH 3.0-3.5 using standard methods such as those described in Harlow and Lane, supra. Fab fragments may be obtained from F(ab′)2 fragments by limited reduction, or from whole antibody by digestion with papain in the presence of reducing agents. Fragments can also be produced by recombinant DNA techniques. Segments of nucleic acids encoding selected fragments are produced by digestion of full-length coding sequences with restriction enzymes, or by de novo synthesis. Often fragments are expressed in the form of phage-coat fusion proteins to provide for affinity-sharpening of antibodies.

To produce antibodies of the invention recombinantly, nucleic acids encoding light and heavy chain variable regions, optionally linked to constant regions, are inserted into expression vectors. The light and heavy chains can be cloned in the same or different expression vectors. The DNA segments encoding antibody chains are operably linked to control sequences in the expression vector(s) that ensure the expression of antibody chains. Such control sequences include a signal sequence, a promoter, an enhancer, and a transcription termination sequence. Expression vectors are typically replicable in the host organisms either as episomes or as an integral part of the host chromosome. E. coli is one procaryotic host particularly useful for expressing antibodies of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which typically contain expression control sequences compatible with the host cell (e.g., an origin of replication) and regulatory sequences such as a lactose promoter system, a tryptophan (trp) promoter system, a beta-lactamase promoter system, or a promoter system from phage lambda. Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired. Mammalian tissue cell culture can also be used to express and produce the antibodies of the present invention (see, e.g., Winnacker, From Genes to Clones VCH Publishers, N.Y., 1987). Eukaryotic cells are preferred, because a number of suitable host cell lines capable of secreting intact antibodies have been developed. Preferred suitable host cells for expressing nucleic acids encoding the immunoglobulins of the invention include: monkey kidney CV1 line transformed by SV40 (COS-7, ATCC CRL 1651); human embryonic kidney line; baby hamster kidney cells (BHK, ATCC CCL 10); Chinese hamster ovary-cells (CHO); mouse sertoli cells; monkey kidney cells (CV1 ATCC CCL 70); african green monkey kidney cells (VERO-76, ATCC CRL 1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); and TRI cells.

The vectors containing the polynucleotide sequences of interest (e.g., the heavy and light chain encoding sequences and expression control sequences) can be transferred into the host cell. Calcium chloride transfection is commonly utilized for prokaryotic cells, whereas calcium phosphate treatment or electroporation can be used for other cellular hosts (see, e.g., Sambrook et al., Molecular Cloning: A Laboratory Manual, Cold Spring Harbor Press, 2nd ed., 1989). When heavy and light chains are cloned on separate expression vectors, the vectors are co-transfected to obtain expression and assembly of intact immunoglobulins. After introduction of recombinant DNA, cell lines expressing immunoglobulin products are cell selected. Cell lines capable of stable expression are preferred (i.e., undiminished levels of expression after fifty passages of the cell line).

Once expressed, the whole antibodies, their dimers, individual light and heavy chains, or other immunoglobulin forms of the present invention can be purified according to standard procedures of the art, including ammonium sulfate precipitation, affinity columns, column chromatography, gel electrophoresis and the like (see, e.g., Scopes, Protein Purification, Springer-Verlag, N.Y., 1982). Substantially pure immunoglobulins of at least about 90 to 95% homogeneity are preferred, and 98 to 99% or more homogeneity most preferred.

By “labeled antibody,” “detectably labeled antibody”” is meant an antibody (or antibody fragment which retains binding specificity), having an attached detectable label. The detectable label is normally attached by chemical conjugation, but where the label is a polypeptide, it could alternatively be attached by genetic engineering techniques. Methods for production of detectably labeled proteins are well known in the art. Detectable labels known in the art, but normally are radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, luciferase/luciferin), methods for labeling antibodies, and methods for using labeled antibodies are well known in the art (see, for example, Harlow and Lane, eds., 1988, Antibodies: A Laboratory Manual, Cold Spring Harbor Laboratory Press, Cold Spring Harbor, N.Y.) Another technique which may also result in greater sensitivity consists of coupling the antibodies to low molecular weight haptens. These haptens can then be specifically detected by means of a second reaction. For example, it is common to use such haptens as biotin, which reacts with avidin, or dinitrophenyl, pyridoxal, and fluorescein, which can react with specific antihapten antibodies.

The antibodies of the invention can be used to detect and/or treat various CPR-associated fibrinolytic and inflammatory disorders in mammalian subjects. The terms CPR-associated fibrinolytic disorder and CPR-associated inflammatory disorder denotes any fibrinolytic or inflammatory disease or condition associated with CPR activity, most commonly involving aberrant expression, metabolism, or activity of CPR (proCPR, activated CPR, and/or inactivated CPR) in the subject. The methods for detecting such disorders generally include contacting a sample from a subject having, or at risk of having, a CPR-associated fribrinolytic or inflammatory disorder with a reagent that detects proCPR, activated CPR, and/or inactivated CPR, and detecting the reaction of the reagent. The reaction of the reagent with the sample is then compared to a control. Any biological sample which may contain a detectable amount of proCPR, activated CPR, and/or inactivated CPR can be used. Examples of biological samples of use with the invention are blood, serum, plasma, urine, mucous, feces, cerebrospinal fluid, pleural fluid, ascites, and sputum samples. Tissue or cell samples can also be used with the subject invention. These samples can be obtained by many methods such as cellular aspiration, or by surgical removal of a biopsy sample. The level of proCPR, activated CPR, and/or inactivated CPR in the sample can be compared with the level in a sample not affected by the targeted disorder or condition. Control samples not affected by a targeted disease processes can be taken from the same subject, or can be from a normal control subject not affected by the disease process, or can be from a cell line.

The methods of the invention for diagnosing fibrinolytic or inflammatory diseases and related conditions involve incubating a reagent that detects proCPR, activated CPR, and/or inactivated CPR with a sample for a time sufficient for the reagent to react with the proCPR, activated CPR, and/or inactivated CPR, and thereafter detecting the reaction of the reagent with the proCPR, activated CPR, and/or inactivated CPR. Within these methods, detection of a reaction is indicative of the presence and/or quantity of proCPR, activated CPR, and/or inactivated CPR in the sample. In certain embodiments, the sample is a patient sample, such as a blood sample, a serum sample, a urine sample, a fecal sample, a tissue biopsy, a cerebrospinal fluid sample, a synovial fluid sample, or a pleural fluid sample. The “reagent that detects proCPR, activated CPR, and/or inactivated CPR” is any molecule that reacts with proCPR, activated CPR, and/or inactivated CPR when incubated therewith. “Reacting” includes binding, such as an antibody binding to an antigen, or the binding of a fluorescent molecule with a binding partner such that the fluorescent properties of a molecule are altered. “Reacting” also includes chemically reacting such that covalent bonds are modified, and includes reacting such that hydrogen bonds are modified. “Incubating” includes conditions which allow contact between the reagent that detects mycothiol or a precursor thereof with a sample. “Contacting” includes in solution and solid phase. “Detection” is performed by any means suitable to identify the interaction of the reagent with proCPR, activated CPR, and/or inactivated CPR. In one embodiment, when the reagent is a chemical reagent, physical or chemical parameters of the reagent or the products of the interaction of the agent with proCPR, activated CPR, and/or inactivated CPR can be monitored. In another embodiment, when the reagent is an antibody, the antibody can be detectably labeled. Detectable labels are well known in the art, and include radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Alternatively, when the reagent is an antibody, detection can be performed using a second antibody which is detectably labeled which recognizes the antibody that binds proCPR, activated CPR, and/or inactivated CPR. The antibody may also be biotinylated, and a second avidinated label used to determine the presence of the biotinylated reagent which detects proCPR, activated CPR, and/or inactivated CPR.

The antibodies of the invention are suited for use, for example, in immunoassays in which they can be utilized in liquid phase or bound to a solid phase carrier. The antibodies employed in these immunoassays can be detectably labeled in various ways. Examples of types of immunoassays which can effectively employ antibodies of the invention are, competitive and non-competitive immunoassays, in either a direct or indirect format. Examples of such immunoassays include a radioimmunoassay (RIA), and a sandwich (immunometric) assay. Those of skill in the art will readily discern additional immunoassay formats useful within the invention.

Other immunoassays for use within the invention include “forward” assays for the detection of a protein in which a first anti-protein antibody (e.g., an anti-proCPR, activated CPR, and/or inactivated CPR anti-antibody) bound to a solid phase support is contacted with the test sample. After a suitable incubation period, the solid phase support is washed to remove unbound protein. A second, distinct anti-protein antibody is then added which is specific for a portion of the specific protein not recognized by the first antibody. The second antibody is preferably detectable. After a second incubation period to permit the detectable antibody to complex with the specific protein bound to the solid phase support through the first antibody, the solid phase support is washed a second time to remove the unbound detectable antibody. Alternatively, the second antibody may not be detectable. In this case, a third detectable antibody, which binds the second antibody is added to the system. This type of “forward sandwich” assay may be a simple yes/no assay to determine whether binding has occurred or may be made quantitative by comparing the amount of detectable antibody with that obtained in a control.

Other types of immunometric assays are the so-called “simultaneous” and “reverse” assays. A simultaneous assay involves a single incubation step wherein the first antibody bound to the solid phase support, the second, detectable antibody and the test sample are added at the same time. After the incubation is completed, the solid phase support is washed to remove unbound proteins. The presence of detectable antibody associated with the solid support is then determined as it would be in a conventional “forward sandwich” assay. The simultaneous assay may also be adapted in a similar manner for the detection of antibodies in a test sample. The “reverse” assay comprises the stepwise addition of a solution of detectable antibody to the test sample followed by an incubation period and the addition of antibody bound to a solid phase support after an additional incubation period. The solid phase support is washed in conventional fashion to remove unbound protein/antibody complexes and unreacted detectable antibody. The determination of detectable antibody associated with the solid phase support is then determined as in the “simultaneous” and “forward” assays. The reverse assay may also be adapted in a similar manner for the detection of antibodies in a test sample.

The antibody component of immunometric assays within the invention may be added to a solid phase support capable of immobilizing proteins. By “solid phase support” or “support” is intended any material capable of binding proteins. Well-known solid phase supports include glass, polystyrene, polypropylene, polyethylene, dextran, nylon, amylases, natural and modified celluloses (including nitrocellulose sheets and filters), polyacrylamides, agaroses, and magnetite. The nature of the support can be either soluble to some extent or insoluble for the purposes of the present invention. The support configuration may be spherical, as in a bead, or cylindrical, as in the inside surface of a test tube or the external surface of a rod. Alternatively, the surface may be flat such as a sheet, test strip, etc. Those skilled in the art will know many other suitable “solid phase supports” for binding proteins or will be able to ascertain the same by use of routine experimentation. A preferred solid phase support is a 96-well microtiter plate. For immunoassay and immunodiagnostic purposes, the antibodies of the invention can be bound to many different carriers, both soluble and insoluble, and can be used to detect the presence of an antigen comprising proCPR, activated CPR, and/or inactivated CPR (or fragments, derivatives, conjugates, homologues, or variants thereof). Those skilled in the art will discern other suitable carriers for binding antibodies useful within the invention. In addition, there are many different labels and methods of labeling known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds, as described above.

In using the antibodies of the invention for the in vitro or in vivo detection of proCPR, activated CPR, and/or inactivated CPR, the detectably labeled antibody is provided in an amount which is diagnostically effective. The term “diagnostically effective” means that the amount of detectably labeled monoclonal antibody is contacted or administered in sufficient quantity to enable detection of proCPR, activated CPR, and/or inactivated CPR in the subject sample to be assayed.

The anti-proCPR, activated CPR, and/or inactivated CPR antibodies of the invention can be used in vitro and in vivo to monitor the appearance, status, course, or treatment of a fibrinolytic or inflammatory disease or condition in a subject. For example, by measuring an increase or decrease in the amount of circulating proCPR, activated CPR, and/or inactivated CPR in a subject (optionally in comparison to control levels in a normal subject or sample), the appearance, status, course, or treatment of the fibrinolytic or inflammatory disease or condition in the subject number can be observed or evaluated. Based on these and comparable diagnostic methods, it is further possible to determine whether a particular therapeutic regimen, such as a treatment regimen employing antibodies of the invention directed against proCPR, activated CPR, and/or inactivated CPR, is effective.

Within more detailed diagnostic methods of the invention, in vivo immunodiagnostic tools are provided, as exemplified by immunoscintigraphic methods and compositions. Immunoscintigraphy (IS) is discussed in detail in P. Lechner et al., Dis Colon Rectum 1993;36:930-935 and F. L. Moffet et al., J Clin Oncol 14:2295-2305 (1966). IS (or radioscintigraphy) employs radioactive-labeled antibody, typically Fab′ fragments (Goldenberg et al.; Eur J Nucl Med 1989;15:426), to recognize defined epitopes of targeted proteins. Fab′ fragments of the antibodies provided herein, comprising immunoglobulins of the IgGI fraction that have their Fc portions removed, are highly capable of targeting epitopes on proCPR, activated CPR, and/or inactivated CPR in a test sample or subject. Because these Fab′ fragments have minimal antigenity, they cause neither human antimouse antibody response, nor any allergic reactions of unpredictable nature. The smaller molecular weight of Fab′ fragments compared with intact antibody allows the fragment to leave the intravascular space and target a broader array of in vivo compartments for diagnostic purposes.

For radioscintigraphy, a radioactive monoclonal antibody of the invention is typically injected into a patient for identifying, measuring, and/or localizing proCPR, activated CPR, and/or inactivated CPR in the subject, (see, e.g., Delaloye et al., Seminars in Nuclear Medicine 25(2):144-164, 1995). In radioimaging with monoclonal antibodies, a chemically modified (chelate) form of the monoclonal antibody is typically prepared and stored as a relatively stable product. To be used clinically, however, the monoclonal antibody sample must be mixed with a radioactive metal, such as ⁹⁹Tc, then purified to remove excess, unbound radioactive metal, and then administered to a patient within 6 hours, (see, e.g., Eckelman et al., Nuc. Med. Biol. 16: 171-176, 1989). Radioisotopes, for example ⁹⁹Tc, an isotope with a short physical half-life and high photon abundance, can be administered at high doses and allow early imaging with a gamma camera. This is very suitable for use in conjunction with Fab′ fragments, the half-lives of which are also short.

Within exemplary embodiments of the invention, diagnostic methods and compositions are provided to assess the status of patients presenting with bacterial infections, anaphylactic conditions, traumatic injury including post-surgical trauma (e.g., following cardiac bypass surgery), and following organ or tissue transplantation. Assays of the invention which detect levels of proCPR, activated CPR, and/or inactivated CPR in a test sample or subject are useful to identify, assess, or quantify inflammatory conditions in these patients, including inflammatory conditions associated with infection, trauma, surgical reaction, and organ or tissue rejection—all of which conditions will be associated with complement activation.

Despite continued improvements in patient and graft survival, several complications may occur in liver transplant patients that lead to graft dysfunction and rejection (Adams et al. (1990) J. Hepatol. 10:113-119). Even though cellular immune responses appear directly responsible for acute allograft rejection, acute and chronic rejection may be enhanced through complement activation. Among other consequences, involvement of complement and generation of activated components in patients with grafted organs may result in enhanced inflammatory responses leading ultimately to severe damage of the organ (Baldwin et al. (1995) Transplantation 59:797-808). Some complement activation data exists for renal transplant patients at the level of tissue C3d and C4d deposition (Feucht et al. (1991) Clin. Exp. Immunol. 86:464-470); Feucht et al. (1993) Kidney Int. 43:1333-1338), and circulating levels of the anaphylatoxins from patients experiencing acute renal disease have been reported (Abou-Ragheb et al. (1991) J. Clin. Lab. Immunol. 35:113-119). In renal and liver transplant patients C3a and C5a levels have been observed to be significantly elevated (Ronholm et al. (1994) Transplantation 57:1594-1597; van Son et al. (1987) Am. Rev. Respir. Dis. 136:580-585).

The methods and compositions of the invention for diagnosis and treatment of patients with inflammatory and fibrionolytic disorders are similarly applicable to immunosuppressed individuals. Immunosuppressed patient are susceptible to recurrent bacterial infections, which in turn primarily activate C3 via the alternative complement pathway (Epstein et al. (1996) Curr. Opin. Immunol. 8:29-35; Reid (1998).

Within additional exemplary embodiments, the methods and compositions of the invention are employed for diagnosis and treatment of patients subjected to extracorporeal circulation (ECC) of the blood, which is a medical procedure used in a variety of life saving medical procedures. Such procedures include hemodialysis, plasmapheresis, plateletpheresis, leukophereses, extracorporeal membrane oxygenation (ECMO) heparin-induced extracorporeal LDL precipitation (HELP), and cardiopulmonary bypass (CPB). Nearly 400,000 CPB surgical procedures are carried out in the United States each year, principally to facilitate coronary artery bypass grafting, but also during other types of open heart surgery, including procedures to correct congenital heart defects, heart valve disease, or other heart defects. Although death during ECC procedures is rare, several acute and chronic complications during and subsequent to these procedures result in potentially life-threatening medical problems and cause significant expense to the health care system. Many of these complications have been associated with activation of the immune system, with the complement arm of the immune system playing a particularly important role in the development of inflammation, platelet dysfunction, thrombocytopenia, and other ECC complications. Activation of the complement system occurs when blood plasma contacts foreign surfaces during ECC. Activated complement components can initiate inflammatory responses, with associated vasoconstriction, capillary leakage and platelet activation.

Alternative diagnostic method to those provided herein often require assessment of pathologic conditions through invasive procedures, such as tissue biopsy (Bronsther et al. (1988) J. Med. Virol. 24:423-434; Snover et al. (1987) Am. J. Surg. Pathol. 11:1-10), causing significant discomfort to the patient, as well as increased expense and considerable risk. A reliable non-invasive detection system as provided herein will obviate these procedures, and will further provide useful methods for monitoring immune status and efficacy of treatments (e.g., status and efficacy of anti-viral, antibiotic, and/or immunosuppressive or immunostimulating treatment regimens). In these and other embodiments, the invention provides diagnostic, monitoring and management methods and compositions, for example, to optimize immunotherapies for transplant patients, and to manage anti-inflammatory care of patients following bacterial infection, or surgery.

The antibodies of the invention can further be employed as therapeutic or prophylactic pharmacological agents in any subject in which it is desirable to administer, in vitro, ex vivo, or in vivo the subject antibodies that bind proCPR, activated CPR, and/or inactivated CPR. Typical subjects for treatment or management according to the methods herein are subjects presenting with a CPR-associated fibrinolytic or inflammatory disorder or condition. Often, the subject will present with a disorder or condition marked by a defect or change in CPR structure, expression, or activity. Most commonly the disorder will involve aberrant expression, metabolism, or activity of CPR, which may be exhibited by one or all endogenous forms of CPR, including proCPR, activated CPR, and/or inactivated CPR in the subject.

In therapeutic embodiments, the selected antibody will typically be a monoclonal antibody, which may be administered alone, or in combination with, or conjugated to, one or more combinatorial therapeutic agents. When the antibodies of the invention are administered alone as therapeutic agents, they may exert a beneficial effect in the subject by a variety of mechanisms. In certain embodiments, monoclonal antibodies that specifically bind a CPR molecule are purified and administered to a patient to neutralize one or more forms of CPR, or to block or inhibit an interaction of one or more forms of CPR with another biomolecule (e.g., a complement protein), and thereby modulate fibrinolytic or inflammatory responses in the subject. In certain embodiments, the antibodies bind to proCPR, activated CPR, and/or inactivated CPR and neutralize or block one or more activities of CPR, which may include neutralization or blockade of CPR interactions with complement proteins.

In some cases, this will include blocking activity of CPR for removing a C-terminal arginine of an anaphylatoxin, which will in turn yield an increase in circulating, activated complement proteins such as C3a, C4a, and C5a anaphylatoxins, and C5b-9 membrane attack complexes. This and related methods will be particularly advantageous for treatment of immuno-compromised patients, and patients with impaired inflammatory capacity. Such pro-inflammatory effects may be directed systemically (e.g., by intravenous or other systemic delivery of the antibody), or locally (e.g., by topical delivery to a wound, skin, or mucosal surface).

In alternate embodiments, the antibodies of the invention will mediate an anti-inflammatory or anti-fibrinolytic response in the subject. Typically, the antibodies will be modified or specially formulated for this purpose. For example, by conjugating the antibodies to a toxin, radionuclide, cross-linking agent, or other chemical moiety as described herein, the antibodies can be modified to neutralize, chelate, cross-link, or otherwise disable or deactivate an anaphylatoxin or membrane attack complex. These effects may be mediated in conjunction with binding of the antibodies to proCPR, activated CPR, and/or inactivated CPR, which binding may include formation of a complex between the modified antibody and CPR, or between the modified antibody, CPR, and a C3a, C4a, or C5a anaphylatoxin, or C5b-9 membrane attack complex. These and other interactions between the antibodies of the invention and one or more targeted, endogenous biomolecules will in turn mediate a change in one or more inflammatory, immune, or fibrinolytic responses or activities in the subject, including a change in the inflammatory, immune, lytic, or fibrinolytic potential of leukocytes, macrophages, vascular tissues, and other cells and tissues, including transplanted cells and tissues.

The immunotherapeutic reagents of the invention may include humanized antibodies, and can be combined for therapeutic use with additional active or inert ingredients, e.g., in conventional pharmaceutically acceptable carriers or diluents, e.g., immunogenic adjuvants, and optionally with adjunctive or combinatorially active agents such as anti-inflammatory ant anti-fibrinolytic drugs.

In other embodiments, therapeutic antibodies of the invention are coordinately administered with, co-formulated with, or coupled to (e.g., covalently bonded) a combinatorial therapeutic agent, for example a radionuclide, a differentiation inducer, a drug, or a toxin. Various known radionuclides can be employed, including ⁹⁰Y, ¹²³I, ¹²⁵I, ¹³¹I, ¹⁸⁶Re, ¹⁸⁸Re, and ²¹¹At. Useful drugs for use in such combinatorial treatment formulations and methods include methotrexate, and pyrimidine and purine analogs. Suitable differentiation inducers include phorbol esters and butyric acid. Suitable toxins include ricin, abrin, diptheria toxin, cholera toxin, gelonin, Pseudomonas exotoxin, Shigella toxin, and pokeweed antiviral protein. These combinatorial therapeutic agents can be coupled to a monoclonal antibody of the invention either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other. Alternatively, it may be desirable to couple a combinatorial therapeutic agent and an antibody via a linker group as a spacer to distance an antibody from the combinatorial therapeutic agent in order to avoid interference with binding capabilities. A linker group can also serve to increase the chemical reactivity of a substituent on an agent or an antibody, and thus increase the coupling efficiency. It will be further evident to those skilled in the art that a variety of bifunctional or polyfunctional reagents, both homo- and hetero-functional (such as those described in the catalog of the Pierce Chemical Co., Rockford, Ill.), may be employed as a linker group. Coupling may be affected, for example, through amino groups, carboxyl groups, sulfhydryl groups or oxidized carbohydrate residues.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.) It may also be desirable to couple more than one agent to an antibody of the invention. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers which provide multiple sites for attachment can be used. Alternatively, a carrier can be used.

A variety of routes of administration for the antibodies and immunoconjugates may be used. Typically, administration is intravenous, intramuscular, or subcutaneous. It will be evident that the precise dose of the antibody/immunoconjugate will vary depending upon such factors as the antibody used, the antigen density, and the rate of clearance of the antibody.

In carrying out various assay, diagnostic, and therapeutic methods of the invention, it is desirable to prepare in advance kits comprises a combination of an antibody of present invention with other materials. For example, in the case of sandwich enzyme immunoassays, kits of the invention may contain a monoclonal antibody that specficially binds proCPR, activated CPR, and/or inactivated CPR, optionally linked to an appropriate carrier, a freeze-dried preparation or a solution of an enzyme-labeled monoclonal antibody which can bind to the same antigen together with the monoclonal antibody or of a polyclonal antibody labeled with the enzyme in the same manner, a standard solution of purified proCPR, activated CPR, and/or inactivated CPR, a buffer solution, a washing solution, pipettes, a reaction container and the like.

The present invention is further illustrated by the following examples which are not intended to limit the effective scope of the claims. All parts and percentages in the examples and throughout the specification and claims are by weight of the final composition unless otherwise specified.

### EXAMPLES

### Production and Characterization of Monoclonal Antibodies Directed Against Carboxypeptidase R

mAbs and ELISA System

The mAbs 2A16 and 10G1 were prepared as described previously (See Guo, et al. (1999) Microbiol. Immunol. 43: 691-698.) The 2A16-coated ELISA plates were prepared by incubating 50 μl of 10 μg/ml 2A16 at 4 C overnight followed by blocking with Block Ace (Yukijirushi, Hokkaido, Japan) and washing with PBS-Tween (PBS containing 0.05% Tween 20) as described elsewhere (See Guo, et al. (1999) Microbiol. Immunol. 43: 691-698.) However, the amount of 2A16 for coating was varied in some experiments as indicated in the Results section. For the second mAb, 10G1 was labeled with horseradish peroxidase (HRP) using a Peroxidase Labeling Kit (Roche, Mannheim, Germany) according to the manufacturer's instructions.

Carboxypeptidase Activity

Carboxypeptidase activity was determined using hippuryl-L-arginine (Hip-Arg) (Peptide Institute Inc. Osaka, Japan) as a substrate which following cleavage, liberates hippuric acid that is then measured by high-pressure liquid chromatography (HPLC) as described previously with slight modifications (See Hendriks, et al. (1985) Clin. Chem. 31: 1936-1939; Watanabe, et al. (1998) Microbiol. Immunol. 42: 393-397.) Briefly, 20 μl samples together with 40 μl HipArg (30 mmol) were incubated at 37° C. for 45 min before addition of 20 μl of 2.5 M HCl to stop the reaction. The hippuric acid released was then extracted with 300 μl ethyl acetate, and 20 μl of the ethyl acetate layer were evaporated and dissolved in 200 μl distilled water for HPLC analysis.

Thrombin and Thrombomodulin Complexes (T-TM)

Thrombin (T) was purchased from Nihon Pharmaceutical Co. Ltd. (Tokyo, Japan) and recombinant thrombomodulin (TM) was generous gift from Asahi Kasei Co. (Tokyo, Japan). Extensive conversion of plasma proCPR into CPR was carried out by addition of T-TM and CaCl₂ generating serum (T-TM serum) according to the method of Schutteman, et al., (See Schutteman et al. (1999) Clin. Chem. 45: 807-813) with some modifications (See Komura, et al. (2002) Microbiol. Immunol. 46: 217-223.) To inhibit the function of thrombin at desired time with a specific inhibitor, Phe-Pro-Arg-chloromethyl ketone (PPACK) purchased from Calbiochem (San Diego, Calif.) was added as described previously (See Komura, et al. (2002) Microbiol. Immunol. 46: 217-223.)

Blood Samples

Blood samples for plasma and serum preparation were taken from healthy colleagues by venipuncture with their agreement.

Amount of proCPR and/or CPR in Plasma, Serum and T-TM Serum

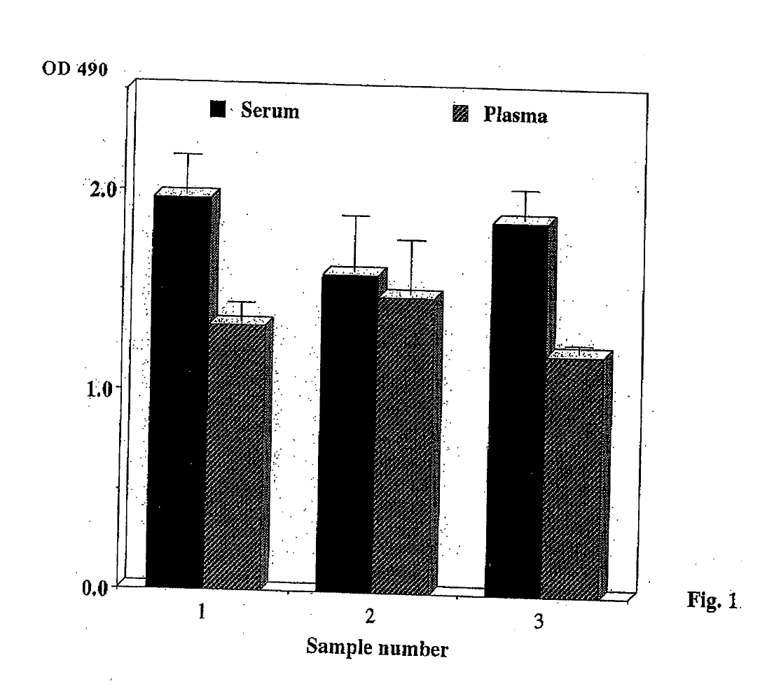

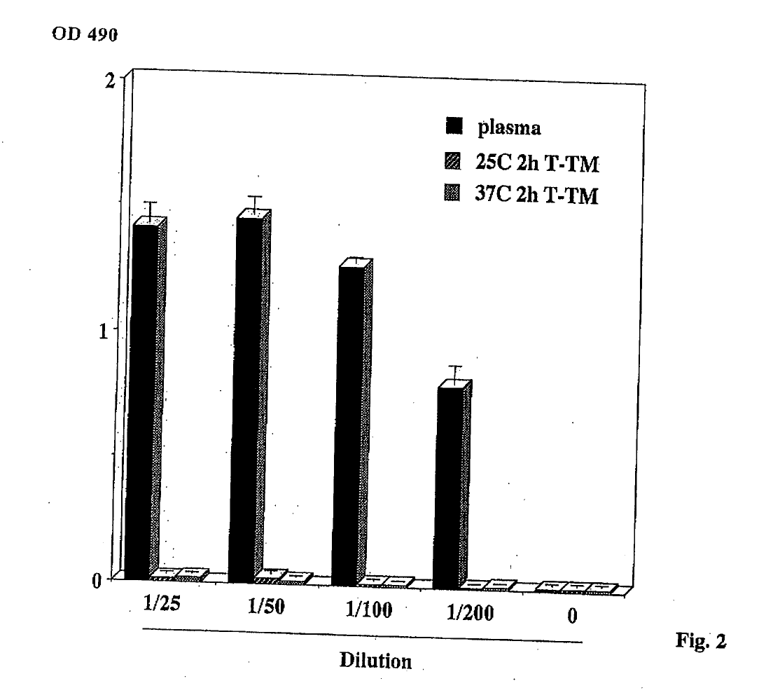

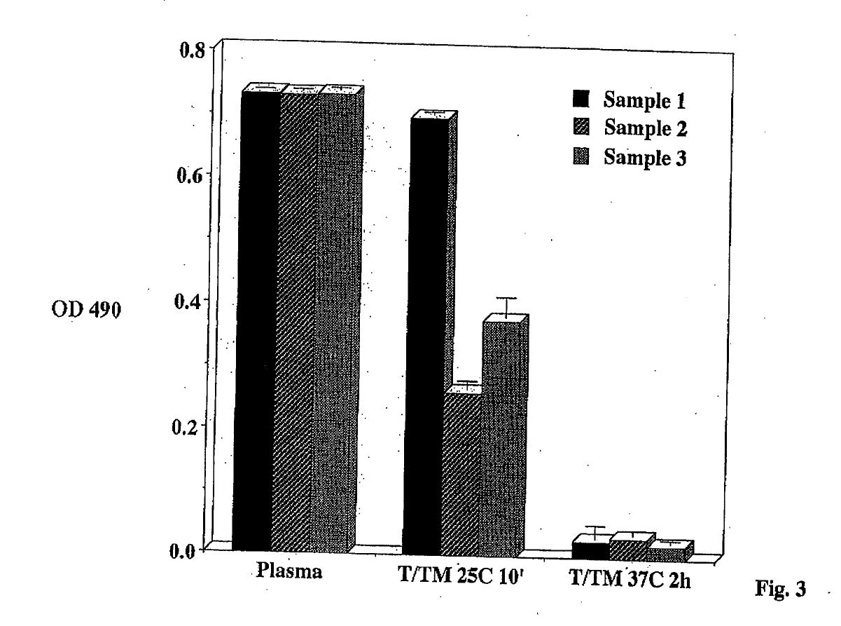

Plasma and serum of 3 healthy individuals were tested for their reactivity on the sandwich ELISA. As shown in FIG. 1, the amount of antigen in serum was significant and even exceeded that in plasma, confirming previous results (See Guo, et al. (1999) Microbiol. Immunol. 43: 691-698.) Since an appreciable amount of proCPR might have remained in serum after activation, extensive conversion of proCPR to CPR was carried out by addition of thrombin and thrombomodulin complexes (T-TM). Plasma was treated with T-TM together with CaCl₂ and incubated at 25° C. or at 37° C. for 2 hr. Although CPR is relatively stable at 25° C. and an appreciable amount of CPR would remain in an active form for a few hr (See Campbell, et al. (1989) Biochem. Biophys. Res. Commun. 162: 933-939; Komura, et al. (2002) Microbiol. Immunol. 46: 217-223), by 2 hr, immunoreactivity in the ELISA system diminished at 25° C. as well as at 37 C (FIG. 2). Plasma treated with T-TM together with CaCl₂ was also incubated at 25° C. for 10 min as well as at 37° C. for 2 hr. Incubation of plasma with T-TM and CaCl₂ at 25° C. for 10 min significantly decreased the immunoreactivity on ELISA of some T-TM serum (FIG. 3). Incubation at 25° C. for 10 min yielded variable results, indicating that the extent of proCPR activation might have been different among the samples for unknown reasons, however this result showed that conversion of proCPR to active CPR might reduce the antigenicity detectable by ELISA because activated CPR is relatively stable at 25° C. and CPR activity does not decrease within 10 min (See Campbell, et al. (1989) Biochem. Biophys. Res. Commun. 162: 933-939; Komura, et al. (2002) Microbiol. Immunol. 46: 217-223.)

Inhibition of 10G1 Binding by Serum With Inactivated CPR

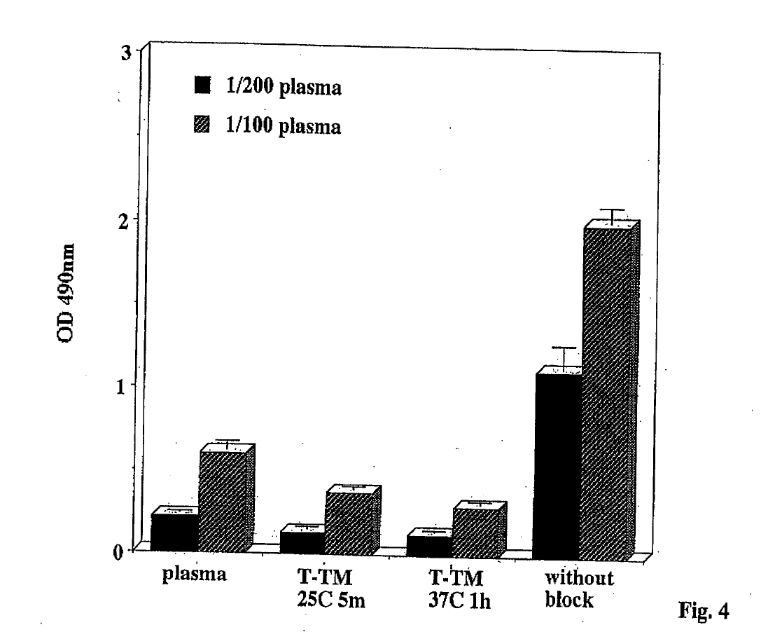

The second antibody, 10G1, which was labeled with HRP (HRP-10G1), was preincubated with plasma, fresh T-TM serum (incubated at 25° C. for 5 min) or inactivated T-TM serum (incubated at 37° C. for 1 hr) and kept at room temperature for 7 hr. The mixtures were applied to a 2A16-coated ELISA plate following treatment with plasma (1/100 or 1/200) at room temperature for 7 hr to capture proCPR. As shown in FIG. 4, 10G1 binding to the plate was blocked even by inactivated T-TM serum indicating that 10G1 reacted with proCPR, active CPR and inactivated CPR. Although the blocking by fresh plasma seemed slightly weaker than by inactivated T-TM serum, a portion of 10G1 antibody that had bound to proPCR in the fluid phase might have been trapped by 2A16 antibody on the plate which failed to bind proCPR during pretreatment with plasma. These results indicate that 10G1 antibody binds to proCPR, activated CPR and inactivated CPR.

2A16 Ab Can React Only With proCPR

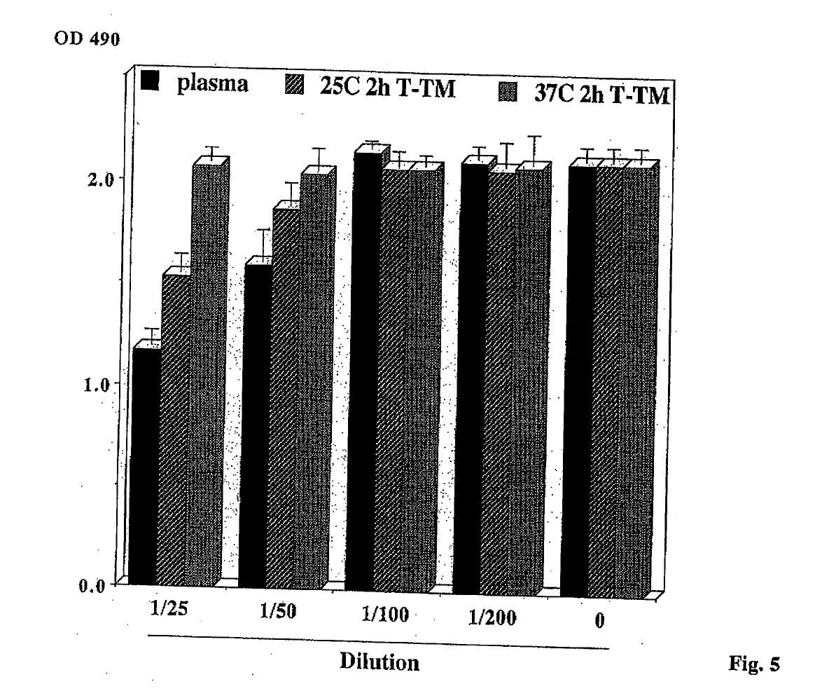

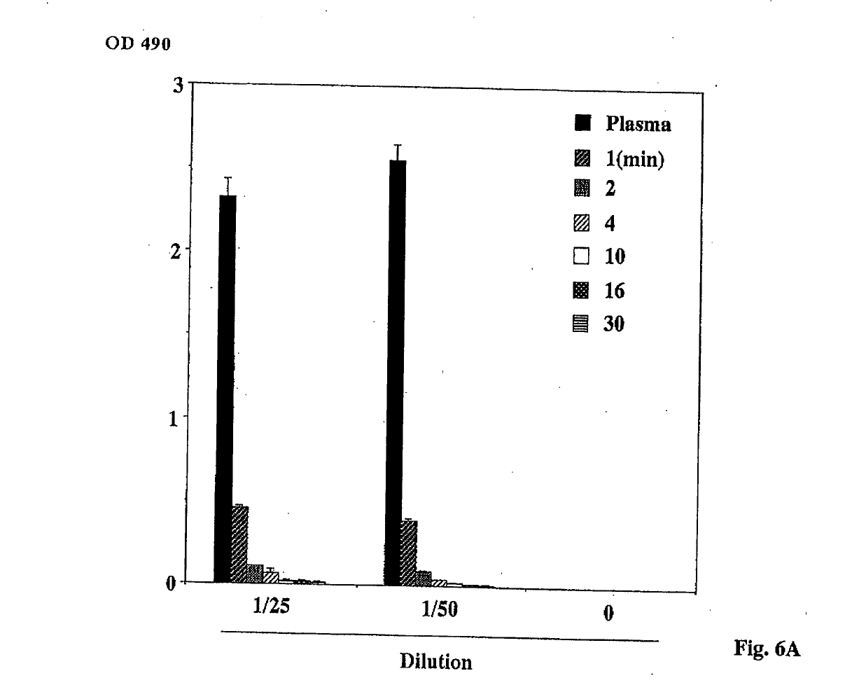

An ELISA plate was coated with 50 μl of 2 μg/ml 2A16. To each well, 50 μl of plasma or T-TM serum incubated for 2 hr (at 25° C. or 37° C.) at dilutions of 1/25, 1/50, 1/100 or 1/200 were added. After overnight incubation at 4° C., each well was treated with a 1/5000 dilution of HRP-10G1 preincubated with 1/200 plasma at 4° C. overnight. Although pretreatment of wells with plasma interfered to some extent with the binding of HRP-10G1 preincubated with plasma to form proCPR-10G1 complexes, T-TM serum scarcely inhibited the binding of the proCPR-10G1 complexes (FIG. 5). This result indicates that 2A16 does not react with inactivated CPR although it may react with fresh CPR to some extent.

Detection of proCPR in Plasma but not CPR in T-TM Serum

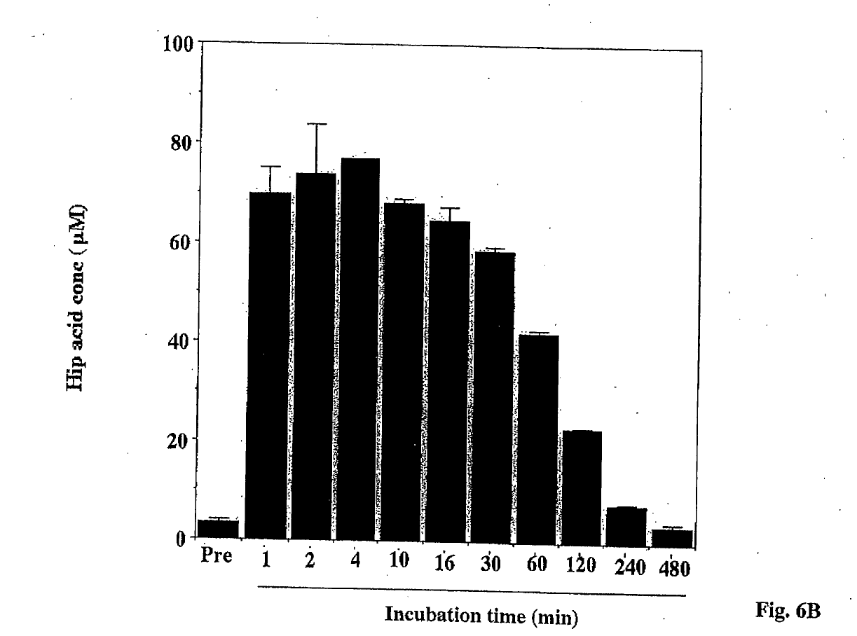

Plasma was converted to serum by addition of T-TM together with CaCl₂ and incubation at 25° C. or 37° C. Incubation was continued for 2 hr and the resulting serum was subjected to the ELISA assay. As shown in FIG. 2, serum generated by T-TM lost antigenicity regardless of the temperature of incubation (25° C. and 37° C.). To determine the time course for loss of the antigenicity, samples from the plasma incubated with T-TM and CaCl₂ at 25° C. were examined by ELISA. The drop in antigenicity started immediately after mixing the plasma with T-TM and a significant reduction in antigenicity was observed over the incubation period (FIG. 6A). The sample at 0 time should have lost antigenicity during incubation for ELISA. By 9 min of incubation, almost all antigenicity was lost. To determine the state of CPR in the T-TM serum, CPR activity was determined and the activity of CPR remained at an appreciable level at 30 min (FIG. 6B). These results indicate that nascently activated CPR has no reactivity with the ELISA system even before inactivation.

Summarizing the foregoing disclosure and examples, ProCPR is a zymogen that is converted to the active form, CPR by trypsin-like enzymes such as thrombin and plasmin (See Bajzar, et al. (1996) J. Biol. Chem. 271: 16603-16608; Campbell, et al. (1990) J. Lab. Clin. Med. 115: 610-642; Eaton, et al. (1991) J. Biol. Chem. 266: 21833-21838; Shinohara, et al. (1991) Int. Arch. Allergy. Immunol. 103: 400-404.) Recently elastase from polymorphonuclear leukocytes has also been shown to activate proCPR (See Kawamura, et al. (2002) Microbiol. Immunol. 46: 225-230.) Since proCPR more efficiently cleaves C-terminal arginine of C-terminal C5a octapeptide than does CPN, proCPR may play a crucial role in inactivation of C5a anaphylatoxin (See Campbell, et al. (2002) Microbiol. Immunol. 46: 131-134; Campbell, et al. (2001) Immunol. Rev. 180: 162-167.) Therefore, the amount of proCPR in plasma is important information for determining the status of patients with inflammatory and/or immunological diseases.

Furthermore, proCPR proved to be the same molecule as TAFI that becomes activated TAFI (TAFIa) and restricts fibrinolysis by removing C-terminal lysine residues from fibrin required for activation of plasminogen (See Bajzar, et al. (1995) J. Biol. Chem. 270: 14477-14484; Bajzar, et al. (1996) J. Biol. Chem. 271: 16603-16608.) Previously, we established a sandwich ELISA system with two kinds of mAbs generated against purified proCPR (See Guo, et al. (1999) Microbiol. Immunol. 43: 691-698.) Since the amounts of antigens detected by the ELISA system in plasma and serum were essentially the same, we concluded that the system detected not only proCPR but also activated and inactivated CPR. Therefore, we termed the antigen detected by the system as CPR-total indicating that it recognizes proCPR, activated CPR and inactivated CPR.

Recently, we discovered that an appreciable amount of proCPR remains in serum and that addition of T-TM promotes further activation of proCPR so as to convert most of the proCPR in plasma (See Bajzar, et al. (1996) J. Biol. Chem. 271: 16603-16608.) Therefore, we reevaluated the sandwich ELISA system with serum converted from plasma by addition of T-TM and CaCl₂.

Before analysis with T-TM for proCPR activation, we determined the immunoreactivity of plasma and serum from 3 individuals with the ELISA system and confirmed our previous observation (See Guo, et al. (1999) Microbiol. Immunol. 43: 691-698) that the reactivity of serum tended to be even higher than that of plasma. This may be due to interference by plasminogen and/or α2-macroglobulin which can bind to proCPR and CPR, respectively (See Eaton, et al. (1991) J. Biol. Chem. 266: 21833-21838; Valnickova, et al. (1996) J. Biol. Chem. 271: 12937-12943.)

We found that the ELISA system does not detect CPR in nascent T-TM-serum indicating that it detects only proCPR. In blocking experiments shown in FIG. 4 and 5, we demonstrated that 2A16 mAb preferentially reacts only with proCPR and not with CPR, although the other mAb (10G1) reacts even with inactivated CPR. With these results, we concluded that the sandwich ELISA system preferentially detects only proCPR and not activated CPR.

To detect proCPR, CPR activity following activation of proCPR by trypsin treatment can be used (See Watanabe, et al. (1998) Microbiol. Immunol. 42: 393-397.) Although the colorimetric assay of CPR activity (See Komura, et al. (2002) Microbiol. Immunol. 46: 115-117) following T-TM activation is a relatively simple method, the sandwich method described here will be much more convenient for detection in clinical laboratories. For detection of proCPR in experimental animals, CPR activity following treatment of plasma with T-TM could be used (See Plummer Jr., et al. (1978) J. Biol. Chem. 253: 3907-3912.) However, an ELISA system to detect proCPR in experimental animals remains to be developed for use in further studies on the in vivo role of proCPR and CPR.

1. A purified, isolated antibody directed against pro-carboxypeptidase R (pro-CPR), which is capable of binding pro-CPR with moderate to high affinity.

2. The purified, isolated antibody of claim 1, wherein the antibody is a monoclonal antibody.

3. The purified, isolated antibody of claim 1, wherein the antibody is a humanized antibody.

4. The purified, isolated antibody of claim 1, which specifically binds to pro-CPR and does not exhibit specific binding against activated CPR or inactivated CPR.

5. The purified, isolated antibody of claim 4, wherein the antibody is a monoclonal antibody.

6. The purified, isolated antibody of claim 4, wherein the antibody is a humanized antibody.

7. A purified, isolated antibody that recognizes all three forms of CPR, including pro-carboxypeptidase R (pro-CPR), activated CPR and inactivated CPR.

8. The purified, isolated antibody of claim 7, wherein the antibody is a monoclonal antibody.

9. The purified, isolated antibody of claim 7, wherein the antibody is a humanized antibody.

10. An immunoassay method comprising the steps of:
contacting a sample containing one or more proteins selected from pro-carboxypeptidase R (pro-CPR), activated CPR, and inactivated CPR with an anti-CPR antibody of claim 1, 4, or 7;
detecting immunoreactivity between said antibody and pro-carboxypeptidase R (pro-CPR), activated CPR, and/or inactivated CPR to determine presence or quantity of pro-carboxypeptidase R (pro-CPR), activated CPR, and/or inactivated CPR in said sample.

11. The immunoassay of claim 10, wherein the antibody specifically binds to pro-CPR and does not exhibit specific binding against activated CPR or inactivated CPR which does not exhibit specific binding against activated CPR or inactivated CPR.

12. The immunoassay of claim 10, wherein the antibody recognizes all three forms of CPR, including pro-carboxypeptidase R (pro-CPR), activated CPR and inactivated CPR

13. The immunoassay of claim 10, which is a sandwich immunoassay further comprising a second antibody which is reactive with said anti-PCR antibody.

14. The immunoassay of claim 10, wherein said antibody is a monoclonal antibody.

15. The immunoassay of claim 10, wherein said antibody is covalently attached to a detectable label.

16. The immunoassay of claim 10, wherein said step of detecting immunoreactivity involves immunoperoxidase staining, immunofluorescence, immunoelectronmicroscopy, or ELISA.

17. A diagnostic method for evaluating the appearance, status, course, or treatment of a fibrinolytic or inflammatory disease or condition in a mammalian subject comprising the steps of:
contacting a biological sample obtained from said subject containing one or more proteins selected from pro-carboxypeptidase R (pro-CPR), activated CPR, and inactivated CPR with an anti-CPR antibody of claim 1, 4, or 7; and
detecting immunoreactivity between said antibody and pro-carboxypeptidase R (pro-CPR), activated CPR, and/or inactivated CPR to determine presence or quantity of pro-carboxypeptidase R (pro-CPR), activated CPR, and/or inactivated CPR in said sample.

18. The diagnostic method of claim 17, wherein the antibody specifically binds to pro-CPR and does not exhibit specific binding against activated CPR or inactivated CPR which does not exhibit specific binding against activated CPR or inactivated CPR.

19. The diagnostic method of claim 17, wherein the antibody recognizes all three forms of CPR, including pro-carboxypeptidase R (pro-CPR), activated CPR and inactivated CPR.

20. The diagnostic method of claim 17, wherein a diagnostic criterion or value is determined based on an increase or decrease in an amount of circulating proCPR, activated CPR, and/or inactivated CPR in the subject compared to a control level(s) of proCPR, activated CPR, and/or inactivated CPR in a normal subject or sample.

21. The diagnostic method of claim 17, wherein said antibody is a monoclonal antibody.

22. The diagnostic method of claim 17, wherein said antibody is covalently attached to a detectable label.

23. The diagnostic method of claim 17, wherein said step of detecting immunoreactivity involves immunoperoxidase staining, immunofluorescence, immunoelectronmicroscopy, or ELISA.

24. The diagnostic method of claim 17, wherein said fibrinolytic or inflammatory disease or condition is selected from or associated with bacterial infection, sepsis, anaphylactic conditions, traumatic injury, post-surgical trauma, extracorporeal circulation (ECC) of the blood, cardiac bypass surgery, organ or tissue transplantation, renal dialysis, leukaphoresis, autoimmune disorders, malignancy, myocardial infarction, and adult respiratory distress syndrome.

25. A method of measuring total carboxypeptidase (CPR) levels in a sample, wherein total CPR includes pro-CPR, activated CPR, and inactivated CPR, comprising the steps of:
contacting the sample with one or more anti-CPR antibody(ies) of claim 1, 4, and/or 7;
detecting immunoreactivity between said antibody(ies) and pro-CPR, activated CPR, and inactivated CPR to determine total pro-CPR, activated CPR, and inactivated CPR in said sample.

26. The method of measuring total CPR of claim 24, further including the step of treating the sample before or after the detecting step to convert pro-CPR to activated CPR, or to convert activated CPR to inactivated CPR.

27. The method of measuring total CPR of claim 24, further including the step of correlating binding of the antibody to a standardized antibody binding profile in order to determine a quantitative value for total CPR in the sample.

28. The method of measuring total CPR of claim 24, further comprising contacting the sample with multiple anti-CPR antibodies of claims 1, 4, and 7, and detecting immunoreactivity between said multiple antibodyies) and pro-CPR, activated CPR, and inactivated CPR to determine total pro-CPR, activated CPR, and inactivated CPR in said sample.
